(19) United States
(12) Patent Application Publication
(10) Pub. No.: US 2026/0234531 A1
(43) Pub. Date: Aug. 13, 2026

(54) SUSPENSION CELL CULTURE BOTTLE

(71) Applicant: CellFabs Inc., Tainan City (TW)

(72) Inventors: Yun-Shu Chiou, Tainan City (TW); Shang-Chih Lin, Tainan City (TW)

(73) Assignee: CellFabs Inc., Tainan City (TW)

(21) Appl. No.: 19/049,387

(22) Filed: Feb. 10, 2025

(51) Int. Cl. C12M 1/00; C12M 1/04; C12M 1/24; C12M 1/34; C12M 3/00

(52) U.S. Cl. CPC C12M 27/20; C12M 23/08; C12M 23/24; C12M 23/26; C12M 23/38; C12M 23/46; C12M 41/26

(57) ABSTRACT

A suspension cell culture bottle includes a container body, multiple guide vanes, and a cap. The container body extends upward from the bottom to form a body portion and a receiving opening. The bottom of the container is equipped with multiple cylindrical structures serving as fixed bases. The base blocks of the guide vanes are inserted into the fixing holes of these cylindrical structures, and the guide vanes are arranged in a helical configuration along the inner wall of the body portion, enhancing internal liquid flow. The cap is fitted onto the receiving opening. When the container is shaken, the guide vanes direct the culture medium upward, thereby improving the shaking effect. This structure enhances cell culture efficiency and improves external air supply for suspension cells, making it suitable for large-scale and customized cell culture applications.

### BACKGROUND OF THE DISCLOSURE

### 1. Field of the Disclosure

The present invention relates to a suspension cell culture bottle, particularly one capable of achieving rapid assembly, reducing costs, and enhancing liquid disturbance functions, thereby significantly improving the efficiency and operational convenience of the cell culture bottle during the cell culture process.

### 2. Description of the Prior Art

Cell culture plays a critical role in the applications of biotechnology, covering areas such as regenerative medicine, vaccine production, and drug development. Currently, conventional cell culture bottles exhibit significant technical limitations in culturing suspension cells. These bottles rely on external forces for shaking to promote the flow of the culture medium, primarily to prevent cell aggregation and enhance contact between the cells and external air. However, due to the simple structure of the bottles, typical shaking only results in horizontal liquid flow, failing to achieve effective vertical disturbance. This leads to uneven cell distribution and low cultivation efficiency.

To address these deficiencies, prior technologies have attempted to improve the internal structure of culture bottles, such as designing specialized curved inner walls to guide the flow of the culture medium, aiming for more uniform fluid disturbance. However, such improvements require precise mold design and manufacturing techniques, and the complex structure of the bottle body may increase production difficulties, significantly raising the production cost of the culture bottles. Although these improvement methods contribute to enhancing fluid circulation, their adoption is limited in practice due to cost considerations. In large-scale cell culture applications, high-cost culture bottles cannot fully replace traditional designs, highlighting the need for solutions that balance performance enhancement and cost control. Therefore, this invention proposes a cell culture bottle with innovative structural and functional designs, effectively reducing production costs and optimizing shaking performance.

In light of these challenges, the inventor, drawing upon years of experience in the manufacturing, development, and design of related products, meticulously designed and assessed solutions to the aforementioned objectives, finally achieving a truly viable invention.

### Summary of the Disclosure

The present invention aims to address the deficiencies of the prior art by providing a suspension cell culture bottle.

A container body extends upward from a container bottom to form a container body section and a receiving opening. The container bottom of the container body is convexly provided with multiple cylinders. Multiple guide vanes are each formed with a base block at one end, and the base block has a fixing hole. The guide vanes are fixedly inserted into the corresponding cylinders through the fixing holes. The guide vanes extend from the base block toward the receiving opening, and multiple guide vanes are helically arranged along the inner wall surface of the container body section. A cap is fitted over the receiving opening, and the center of the cap is provided with a cap opening, wherein a cover plate is pivotally connected to the cap opening.

The cap is equipped with a resistor at the cover plate. At least one window is formed around the periphery of the cap opening, and the window is covered with a breathable waterproof membrane. The breathable properties of the breathable waterproof membrane allow air to circulate into the interior of the container body.

The cap is provided with an inspection port, and a pH sensor is fixed to the exterior of the cap. The pH sensor includes a sensor rod, which is inserted into the inspection port and extends into the interior of the container body.

The pH sensor is electrically connected to a processor, a power supply, and a WiFi communicator. The power supply provides the required power to the processor, and the processor is used to interpret the pH data from the sensor rod, which is then wirelessly transmitted via the WiFi communicator.

The container body is transparent, and the container bottom of the container body is recessed inward to form multiple positioning recesses.

The container body is formed with a clamping section between the container body section and the receiving opening, and the clamping section is planar, providing an interface for external tools to clamp and move the bottle.

A clamping section is convexly formed at the center of the container bottom, and the clamping section is fitted with a flow guide component, which guides the internal fluid of the container body from the center toward the direction of the guide vanes.

The clamping section is annularly provided with multiple elastic pillars in a convex arrangement. The top of each elastic pillar is formed with a protruding blocking piece. The flow guide component comprises a ring body and multiple blades arranged around the ring body. The flow guide component is fitted over all the elastic pillars through the ring body, and the blocking pieces prevent the ring body from moving, thus positioning the flow guide component.

Each elastic pillar forms a protrusion adjacent to the container bottom, which supports the ring body of the flow guide component, creating multiple flow openings between the ring body and the elastic pillars.

At least one sphere is attached to the blades of the flow guide component. The sphere contacts the container bottom of the container body, and both the blades and the sphere of the flow guide component can rotate around the clamping section.

The aforementioned structural design enables rapid assembly, cost reduction, and enhanced liquid disturbance functionality, significantly improving the efficiency and operational convenience of the cell culture bottle during the cell culture process.

Other objectives, advantages, and novel features of the present invention will become more apparent from the following detailed description and accompanying drawings.

### BRIEF DESCRIPTION OF THE DRAWINGS

The present invention will be best understood by referring to the following detailed description of one illustrative embodiment in conjunction with the accompanying drawings, in which

FIG. 1 is a perspective view of the present invention;

FIG. 2 is an exploded perspective view of the present invention;

FIG. 3 is a bottom view of the cap of the present invention;

FIG. 4 is a side view of the present invention;

FIG. 5 is a schematic view showing the shaking state of the present invention;

FIG. 6 is a schematic diagram of the component relationships of the pH sensor of the present invention;

FIG. 7 is an exploded perspective view of the clamping section and flow guide component of the present invention;

FIG. 8 is a schematic view showing the shaking state of the flow guide component of the present invention;

FIG. 9 is a schematic view illustrating the liquid replacement operation of the present invention.

### DETAILED DESCRIPTION

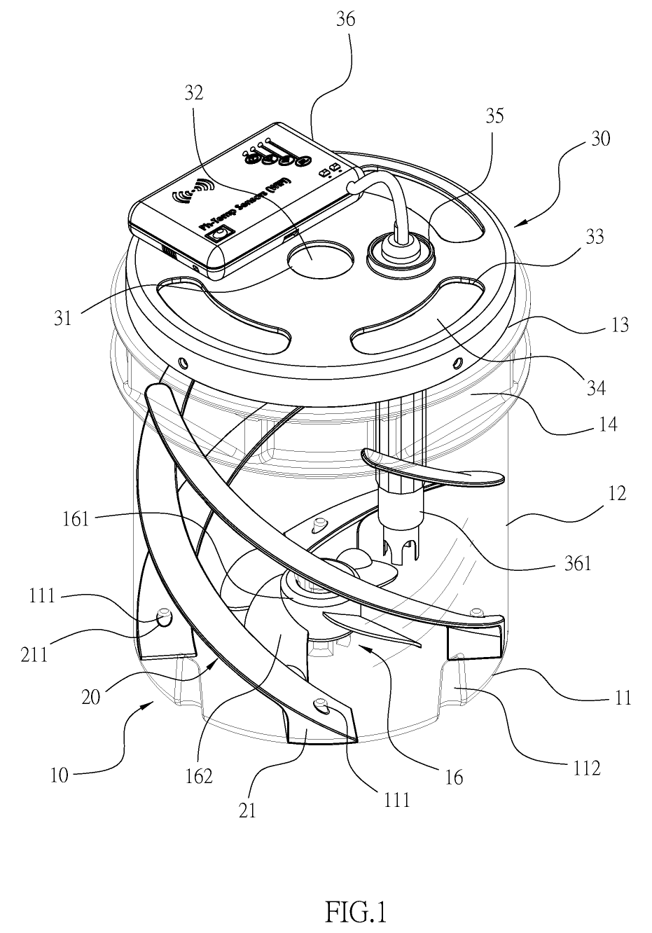

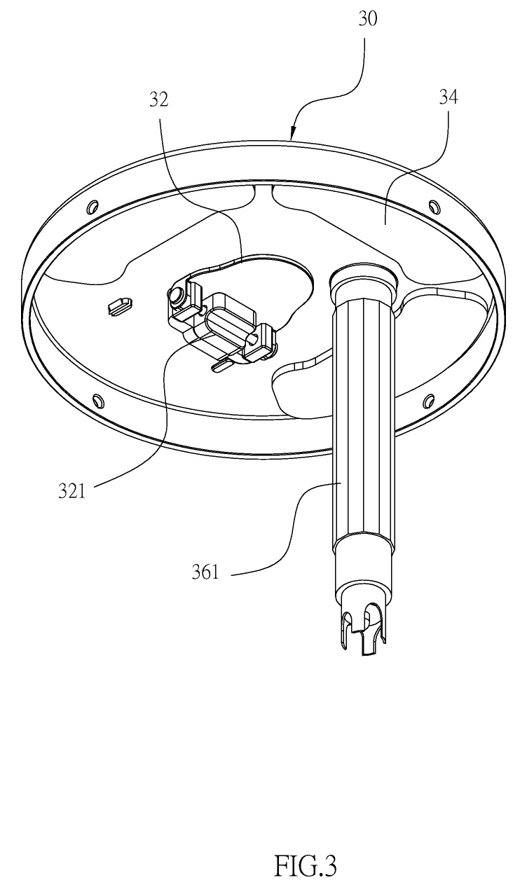

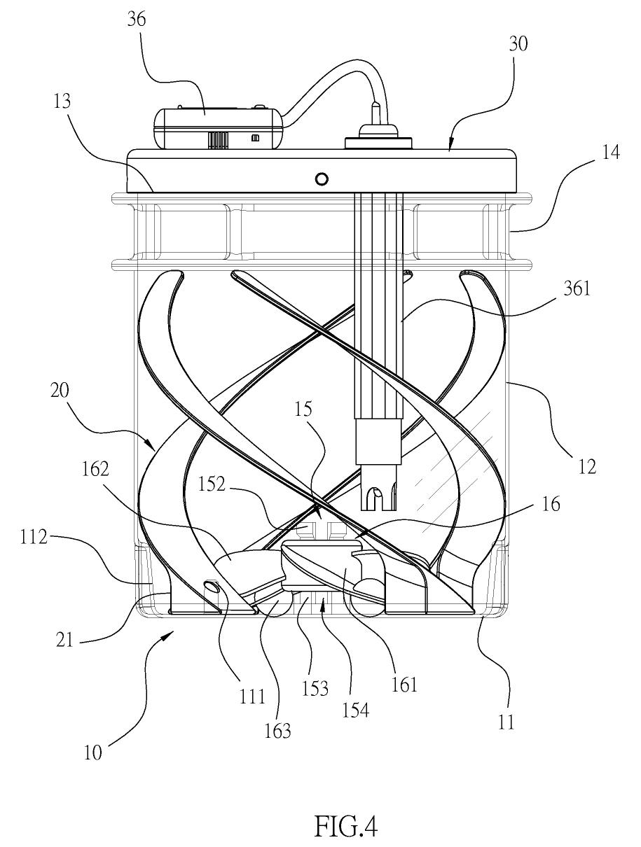

Referring to FIG. 1 through 5, a suspension cell culture bottle includes a container body 10, a plurality of guide vanes 20, and a cap 30. The container body 10 extends upward from a container bottom 11 to form a container body section 12 and a receiving opening 13. The container bottom 11 is inwardly convex and provided with a plurality of cylinders 111, which are evenly distributed on the container bottom 11. Additionally, the container bottom 11 has a plurality of inwardly recessed positioning recesses 112 for securely connecting the container body 10 to a shaking mechanism (not shown in the figures), preventing tipping or detachment during the shaking process.

The container body section 12 extends upward from the container bottom 11, forming a cylindrical structure. Its inner wall is circumferentially arranged with a plurality of guide vanes 20. Each guide vane 20 has a base block 21 at one end, which is provided with a fixing hole 211. The fixing hole 211 is embedded in the cylinder 111 to achieve a secure connection. Each guide vane 20 extends from the base block 21 toward the receiving opening 13, and all the guide vanes 20 are arranged in a helical pattern, forming a vortex flow structure. During shaking, the culture liquid flows outward under the influence of centrifugal force, is directed upward along the guide vanes 20, and recirculates near the receiving opening 13 back to the center of the container body 10. This design enables vertical fluid agitation, preventing cell clumping, enhancing gas-liquid exchange efficiency in the culture liquid, and further promoting cell growth.

The receiving opening 13 is located at the top of the container body 10 for connection with the cap 30. The center of the cap 30 has a cap opening 31, which is pivotally connected to a cover plate 32 for convenient sealing or opening of the receiving opening 13. The cap 30 is equipped with a resistor 321 at the cover plate 32, which provides a slow-return function. This allows the cover plate 32 to close slowly after being opened, thereby reducing the adhesion of culture liquid on the cover plate 32. The cap opening 31 is used for extracting and injecting culture liquid during replacement.

The periphery of the cap 30 is provided with at least one window 33, and each window 33 is fitted with a breathable waterproof membrane 34. The breathable waterproof membrane 34 allows free air circulation while preventing moisture penetration, ensuring gas exchange in the culture environment, avoiding liquid spillage and bacterial contamination, and improving the stability of the culture process.

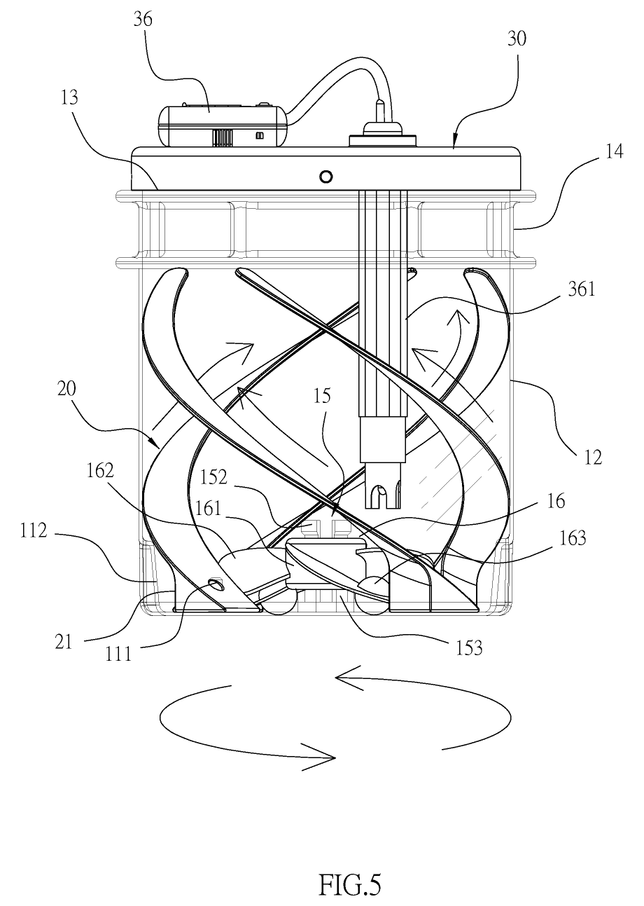

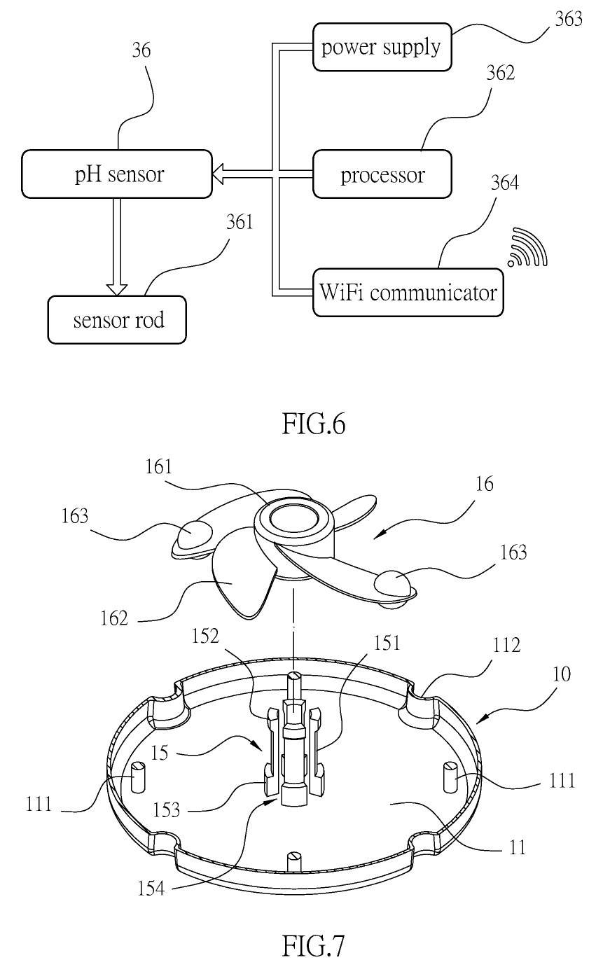

Further elaboration, as shown in FIGS. 1, 2, and 6, in the embodiment, the cap 30 is provided with an inspection port 35, into which a pH sensor 36 is inserted. The pH sensor 36 comprises a sensor rod 361, a processor 362, a power supply 363, and a WiFi communicator 364. The sensor rod 361 extends from the inspection port 35 into the interior of the container body 10 and is used to monitor the pH value of the culture liquid in real time. The data collected by the sensor rod 361 is analyzed by the processor 362 and transmitted to external devices via the WiFi communicator 364. This design allows users to monitor changes in the pH of the culture environment without opening the container body 10 and to adjust the shaking time or perform liquid replacement operations based on the data.

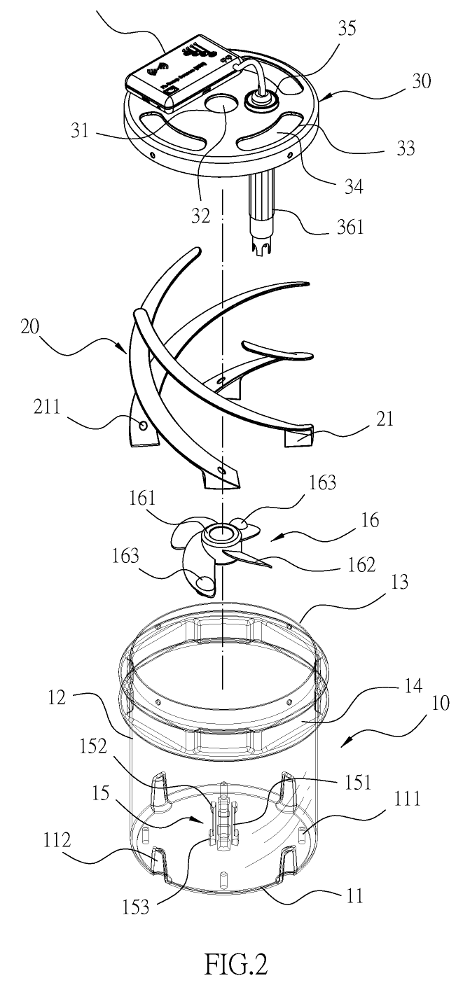

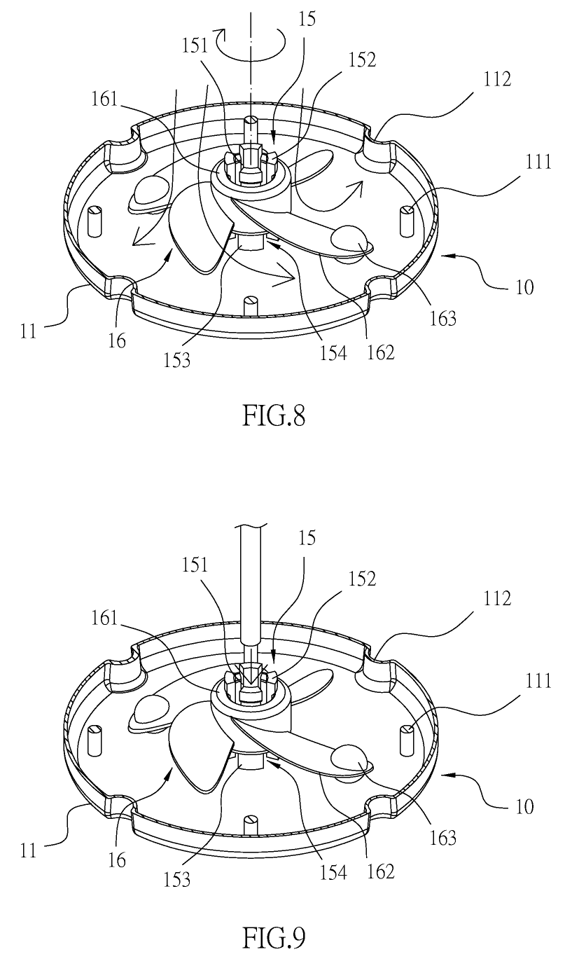

Referring to FIGS. 7, 8, and 9 in conjunction with FIG. 2, the center of the container bottom 11 is provided with a clamping section 15. The clamping section 15 is formed with multiple elastic pillars 151 arranged in a circular manner. Each elastic pillar 151 has a convex-shaped blocking piece 152 at its top end. A flow guide component 16 is sleeved onto the clamping section 15. The flow guide component 16 comprises a ring body 161 and multiple blades 162 arranged around the ring body 161. The ring body 161 is sleeved onto the elastic pillars 151 and constrained by the blocking pieces 152 to prevent detachment while maintaining the rotatable functionality of the flow guide component 16. The blades 162 and the ring body 161 collectively form a fluid guiding structure. This design enables the quick assembly of the flow guide component 16 and allows it to freely rotate within the constrained range, enhancing the fluid flow performance of the cell culture liquid.

Additionally, adjacent to the container bottom 11, the elastic pillars 151 are formed with protrusions 153. These protrusions 153 support the ring body 161 of the flow guide component 16, forming multiple flow openings 154 between the ring body 161 and the elastic pillars 151. During liquid replacement operations, the culture liquid can be extracted through the center of the clamping section 15, and the flow openings 154 allow the liquid deposited at the container bottom 11 to flow toward the center of the clamping section 15 for extraction. This design ensures that the culture liquid at the container bottom 11 can be effectively replaced, preventing residues from affecting the quality of subsequent cell cultures.

Furthermore, the blades 162 are coupled with at least one sphere 163, which contacts the container bottom 11 and can slide along its surface. When the flow guide component 16 rotates, the blades 162 drive the sphere 163 to slide along the container bottom 11, effectively disturbing the sediments deposited on the container bottom 11 and resuspending them into the culture liquid. This design combines the fluid-guiding functionality of the blades 162 and the mobility of the sphere 163, further enhancing the uniformity and hydrodynamic efficiency of the culture liquid.

To further enable automated operations, a clamping section 14 is formed between the container body section 12 and the receiving opening 13 of the container body 10. The clamping section 14 has a flat structure designed to cooperate with external clamping devices, allowing the container body 10 to be precisely moved and positioned through clamping operations.It should be realized that the above description is only some preferred embodiments of the present invention and should not be deemed as limitations of implementing the present invention. All substantially equivalent variations and modifications which employ the concepts disclosed in this specification and the appended claims should fall within the scope of the present invention.

What is claimed is:

1. A suspension cell culture bottle, comprising:
a container body, which extends upward from a container bottom to form a container body section and a receiving opening, wherein the container bottom of the container body is convexly formed inward with multiple cylinders;
multiple guide vanes, each guide vane has a base block at one end, and the base block is provided with a fixing hole, the guide vane is fixed to the cylinder through the fixing hole, and the guide vane extends from the base block toward the receiving opening, multiple guide vanes are helically arranged along the inner wall of the container body section;
a cap, which is fitted to the receiving opening, the center of the cap has a cap opening, and a cover plate is pivotally connected at the cap opening.

2. The suspension cell culture bottle of claim 1, wherein the cap is equipped with a resistor at the cover plate, and at least one window is formed around the periphery of the cap opening, a breathable waterproof membrane is attached to the window, and the breathable characteristics of the breathable waterproof membrane allow air to flow into the container body.

3. The suspension cell culture bottle of claim 1, wherein the cap is provided with an inspection port, and a pH sensor is fixed externally to the cap, the pH sensor includes a sensor rod, which is inserted into the inspection port and extends into the container body.

4. The suspension cell culture bottle of claim 3, wherein the pH sensor is electrically connected to a processor, a power supply, and a wifi communicator, the power supply provides power for the processor, which interprets the pH data from the sensor rod, and the wifi communicator enables wireless data transmission.

5. The suspension cell culture bottle of claim 1, wherein the container body is transparent, and the container bottom of the container body is recessed inward to form multiple positioning recesses.

6. The suspension cell culture bottle of claim 1, wherein the container body forms a clamping section between the container body section and the receiving opening, and the clamping section is planar to provide clamping and movement by external instruments.

7. The suspension cell culture bottle of claim 1, wherein the container body has a clamping section protruding at the center of the container bottom, and the clamping section is fitted with a flow guide component, allowing the fluid inside the container body to be directed centrally towards the guide vane.

8. The suspension cell culture bottle of claim 7, wherein the clamping section is annularly protruded with multiple elastic pillars, and the top of each elastic pillar forms an outwardly protruding blocking piece, the flow guide component includes a ring body and multiple blades surrounding the ring body, the flow guide component is fitted onto all the elastic pillars via the ring body, and the blocking piece restricts the ring body to limit the flow guide component's movement.

9. The suspension cell culture bottle of claim 8, wherein a protrusion is formed adjacent to the container bottom at the elastic pillar, supporting the ring body of the flow guide component and forming multiple flow openings between the ring body and the elastic pillar.

10. The suspension cell culture bottle of claim 8, wherein at least one sphere is attached to the blade, and the sphere contacts the container bottom of the container body, The blades and the sphere of the flow guide component can both rotate around the clamping section.
